(19) United States
(12) Patent Application Publication
(10) Pub. No.: US 2024/0152597 A1
(43) Pub. Date: May 9, 2024

(54) ROBOT DEVICE, SURGICAL MANIPULATOR, AND SYSTEM

(71) Applicant: SONY GROUP CORPORATION, TOKYO (JP)

(72) Inventor: HIROYUKI SUZUKI, TOKYO (JP)

(21) Appl. No.: 18/548,667

(22) PCT Filed: Dec. 28, 2021
(86) PCT No.: PCT/JP2021/049008

§ 371 Date: Sep. 1, 2023

(30) Foreign Application Priority Data
Mar. 9, 2021 (JP) 2021-037863

(51) Int. Cl. G06F 21/44; A61B 34/00; A61B 34/30; B25J 17/00; B25J 19/00; H05K 1/02; H05K 1/11; H05K 1/14

(52) U.S. Cl. CPC G06F 21/44; A61B 34/30; A61B 34/70; B25J 17/00; B25J 19/0029; H05K 1/0278; H05K 1/118; H05K 1/147; H05K 1/0274

(57) ABSTRACT

Provided is a robot device having a structure in which a plurality of links is hinge-coupled, and having simplified wiring for signal and power transmission at a hinge portion.

The robot device includes a plurality of links and a hinge part that includes a deformable signal transmission part and connects the links adjacent to each other. The robot device further includes a flexible circuit board including a signal transmission line layer and a low-rigidity insulating layer stacked on top of each other, the signal transmission line layer transmitting a signal, the low-rigidity insulating layer insulating the signal transmission line layer, each of the plurality of links is formed by the flexible circuit board having a high-rigidity material bonded to both sides or at least one side thereof, and the hinge part is formed by the flexible board having no high-rigidity material bonded to either of the sides thereof.

### TECHNICAL FIELD

The technology disclosed herein (hereinafter, “the present disclosure”) relates to a robot device, a surgical manipulator, and a system having a plurality of links hinge-coupled.

### BACKGROUND ART

Recent surgical systems have made use of a robotics technology mainly for prevention of a tremor in hands of an operator, operation support, absorption of a difference in skill between operators, implementation of remote surgery, and the like.

Here, for a surgical manipulator having a configuration in which a plurality of links is hinge-coupled, it is necessary to route wiring for signal and power transmission, so that the following problems will occur.
(1) Structural layout in mechanical design is greatly restricted.
(2) Wire rigidity adversely affects a control system while a robot is in operation.
(3) Difficulty in assembly increases.
(4) There is a risk of false connection of wiring when the end effector is attached.
(5) Difficulty in satisfying medical requirements such as cleaning and sterilization increases.

For example, a method for producing a three-dimensional structure that develops into a three-dimensional shape like paper-folding by laminating a plurality of layers on a single sheet and partially cutting the laminate has been proposed (see Patent Document 1). Robot manufacturing is simplified on the basis of the production method; on the other hand, in a case where a drive unit is mounted on a distal end located away from a mechanical ground, it is necessary to route electric wiring from the outside, and thus, there is concern that control performance is adversely affected by rigidity and tension of the electric wiring.

### CITATION LIST

### Patent Document

Patent Document 1: Japanese Translation of PCT Application No. 2014-512973

### SUMMARY OF THE INVENTION

### Problems to be Solved by the Invention

It is therefore an object of the present disclosure to provide a robot device having a structure in which a plurality of links is hinge-coupled, and having simplified wiring for signal and power transmission at a hinge portion, a surgical manipulator that has a link structure and to which a surgical instrument is attached, and a system.

### Solutions to Problems

The present disclosure has been made in view of the above-described problems, and a first aspect of the present disclosure is a robot device including: a plurality of links; and a hinge part including a deformable signal transmission part and connecting the links adjacent to each other.

The robot device according to the first aspect further includes a flexible circuit board including a signal transmission line layer and a low-rigidity insulating layer stacked on top of each other, the signal transmission line layer transmitting a signal, the low-rigidity insulating layer insulating the signal transmission line layer. Then, each of the plurality of links is formed by the flexible circuit board having a high-rigidity material bonded to both sides or at least one side thereof. Furthermore, the hinge part is formed by the flexible board having no high-rigidity material bonded to either of the sides thereof.

The signal transmission line layer includes a conductive layer that transmits an electric signal. Then, in a case where the robot device according to the first aspect includes an open link structure, the open link structure includes an electrode pad used for transmission and reception of the electric signal provided at both ends of the flexible board, the electrode pad being formed by an exposed portion of the signal transmission line layer. Furthermore, in a case where the robot device according to the first aspect includes a closed link structure, at least some of the high-rigidity materials bonded to the links have an opening, and the closed link structure includes an electrode pad used for transmission and reception of the electric signal, the electrode pad being formed by a portion of the signal transmission line layer exposed through the opening.

Furthermore, the robot device according to the first aspect may include a plurality of the closed link structures coupled to each other.

Furthermore, a second aspect of the present disclosure is a surgical manipulator including: a surgical instrument; and a link structure including a plurality of links and a hinge part including a deformable signal transmission part and connecting the links adjacent to each other, in which the surgical instrument is attached to a link located at a distal end.

The link structure may cause the surgical instrument to pivot with a predetermined trocar insertion point on an axis of the surgical instrument fixed.

Furthermore, a third aspect of the present disclosure is a system including: a robot device including a plurality of links and a hinge part that includes a deformable signal transmission part and connects the links adjacent to each other, an end effector being attached to a link located at a distal end; and an authentication server configured to perform authentication of the end effector, in which the robot device transmits identification information read from the end effector via the signal transmission part to the authentication server, and the authentication server performs authentication processing on the end effector on the basis of the identification information received from the robot device, and acquires configuration data for the end effector.

Note that a “system” described herein refers to a logical assembly of a plurality of devices (or functional modules that implement specific functions), and each of the devices or functional modules may be or may be not in a single housing.

### Effects of the Invention

According to the present disclosure, it is possible to provide a robot device having a structure in which a plurality of links is hinge-coupled and allowing simple routing of wiring for signal and power transmission by routing the wiring through a hinge, a surgical manipulator that has a link structure allowing simple routing of wiring and to which a surgical instrument is attached, and a system that performs processing such as authentication of the surgical instrument attached to the surgical manipulator.

Note that the effects described herein are merely examples, and the effects brought about by the present disclosure are not limited thereto. Furthermore, the present disclosure may further provide additional effects in addition to the effects described above.

Still another object, feature, and advantage of the present disclosure will become clear by further detailed description with reference to an embodiment to be described later and the attached drawings.

### BRIEF DESCRIPTION OF DRAWINGS

FIG. 1 is a diagram depicting a cross-sectional configuration example of an electric circuit board 100 applied to the present disclosure.

FIG. 2 is a diagram depicting an example of an open link structure 200 formed using an FCB.

FIG. 3 is a diagram depicting an example of a closed link structure 300 formed using an FCB.

FIG. 4 is a diagram depicting another example of an open link structure 400 formed using an FCB.

FIG. 5 is a diagram depicting another example of a closed link structure 500 formed using an FCB.

FIG. 6 is a diagram depicting a degree-of-freedom configuration of the closed link structures depicted in FIGS. 3 and 5.

FIG. 7 is a diagram depicting an operation example of a degree-of-freedom configuration model 600 including a parallel link mechanism.

FIG. 8 is a diagram depicting an operation example of the degree-of-freedom configuration model 600 including a parallel link mechanism.

FIG. 9 is a diagram depicting a configuration example of a manipulator 900.

FIG. 10 is a diagram depicting a degree-of-freedom configuration model of the manipulator 900.

FIG. 11 is a diagram depicting the degree-of-freedom configuration model of the manipulator 900.

FIG. 12 is a diagram depicting an example where a surgical instrument is used with the surgical instrument attached to the manipulator 900.

FIG. 13 is a diagram depicting a configuration example of a system 1300 in which the manipulator 900 acquires surgical instrument information.

FIG. 14 is a diagram depicting an example of a three-dimensional image of a manipulator 1400.

### MODE FOR CARRYING OUT THE INVENTION

Hereinafter, the present disclosure will be described in the following order with reference to the drawings.
A. Overview
B. Basic Configuration
C. Configuration of Manipulator
D. Kinematics of Manipulator
E. Usage Example of Surgical Instrument
F. Effects
G. Modification Example

### A. Overview

One major cause of difficulty in wiring in a manipulator is that a hinge structure is based on a rotation structure with a pin as an axis. In a case where a joint (rotation axis) and a link are connected in series or in parallel as in a multi-degree-of-freedom robot arm, wiring to an end effector connected to a distal end of the arm is designed to pass through the rotation axis and the center of the link to the extent possible. However, the closer the wiring is to the center, the greater difficulty in assembly or disassembly, which increases a manufacturing cost or a risk of failure.

Aerial wiring corresponding to wiring made away from the link, such as an industrial robot arm, improves serviceability for assembly and disassembly, but has a risk of adversely affecting control performance due to a decrease in cable rigidity and a risk of cutting the wiring by mistake when a user operates a product. In addition, in a case where a plurality of types of end effectors is changed and operated, it is necessary to make wiring every time an end effector is changed, which makes a workload excessively large.

Furthermore, in a case where a surgical manipulator is used, in order to ensure cleanliness of a surgical instrument that serves as an end effector, it is necessary to clean and sterilize the surgical instrument every time the surgical instrument is changed, and to structurally separate a clean region and a non-clean region. If the wiring is complicated, the sterilization process becomes difficult. Furthermore, given that a medical worker who may not be familiar with electric wiring performs the work of changing the surgical instrument, it is necessary to make the wiring structure easy to understand and simple.

Therefore, the present disclosure proposes a surgical manipulator having a wiring structure that includes a plurality of links, allows easy change of an end effector (for example, a surgical instrument) mounted on a tip (or a distal end), and allows structural separation of a clean region and a non-clean region. As will be described later, the surgical manipulator according to the present disclosure includes a new wiring structure passing through the inside of a hinge connecting links, and a hardware and system configuration that allows easy switching between a plurality of end effectors.

### B. Basic Configuration

The surgical manipulator according to the present disclosure forms a plurality of links and a hinge connecting the links using a flexible electric circuit board having low rigidity and flexibility. With such a basic configuration, it is possible to realize a wiring structure passing through a hinge.

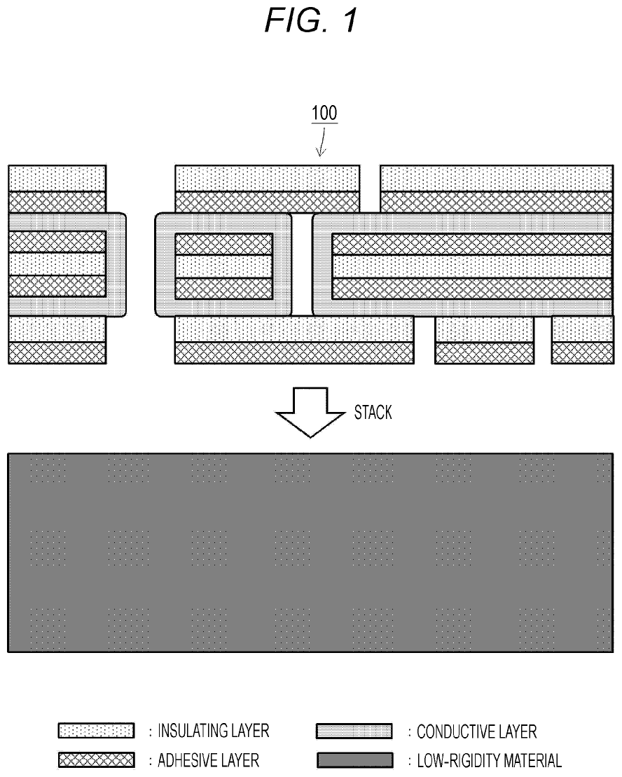

FIG. 1 depicts a cross-sectional configuration example of an electric circuit board 100 applied to the present disclosure. As can be seen from the drawing, the electric circuit board 100 has a multilayer structure including a plurality of pairs of insulating layers and conductive layers, each pair being bonded together with an adhesive layer, the insulating layer including a high-electron polymer or polyimide, and the conductive layer formed by a deposited metal such as copper or aluminum. The multilayer structure has several through holes formed therethrough, the through holes connecting the conductive layers across a plurality of layers. A method for manufacturing the electric circuit board 100 having such a multilayer structure is not limited to any specific method. For example, examples of the method include a method in which the adhesive layer is provided on the conductive layer prepared in advance, and the insulating layer and the conductive layer are bonded together.

Then, finally, a surface of the multilayer structure including the insulating layer, the conductive layer, and the adhesive layer is covered with a low-rigidity material including polyimide or the like, thereby forming the electric circuit board 100 having low rigidity and flexibility. Herein, an electric circuit board having such a multilayer structure and having low rigidity and flexibility is also referred to as a flexible circuit board (FCB). The FCB may be the same as general flexible printed circuits (FPCs).

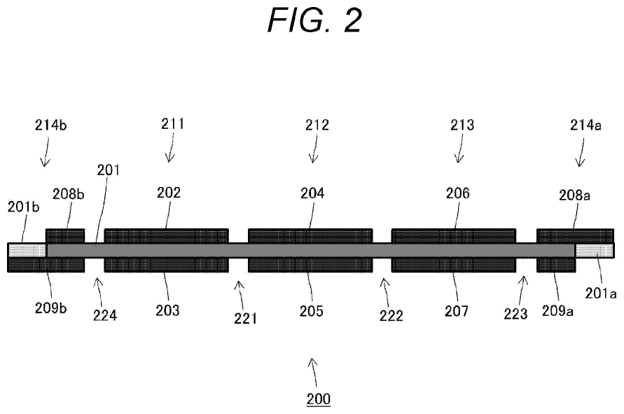

FIG. 2 depicts an example of an open link structure 200 formed using the FCB. In the illustrated open link structure 200, with a low-rigidity FCB 201 located at a center of the open link structure 200, a link 211 with high rigidity can be formed by bonding a pair of high-rigidity parts 202 and 203 including a high-rigidity material that is higher in rigidity than the FCB 201 to front and back surfaces of the FCB 201. In FIG. 2, the FCB 201 is depicted in a simplified manner, but actually has a cross-sectional structure as depicted in FIG. 1. Examples of the high-rigidity material include metals such as titanium, stainless steel, and iron, and ceramics such as carbon and alumina. Furthermore, examples of a method for bonding the front surface and the back surface of the FCB 201 and the high-rigidity parts 202 and 203 include thermal press, adhesion, and the like. It goes without saying that other bonding methods may be used.

Similarly, a link 212 with high rigidity can be formed by bonding a pair of high-rigidity parts 204 and 205 to the front and back surfaces of the FCB 201, a link 213 with high rigidity can be formed by bonding a pair of high-rigidity parts 206 and 207 to the front and back surfaces of the FCB 201, a link 214a with high rigidity can be formed by bonding a pair of high-rigidity parts 208a and 209a to the front and back surfaces of the FCB 201, and a link 214b with high rigidity can be formed by bonding a pair of high-rigidity parts 208b and 209b to the front and back surfaces of the FCB 201. Note that the links 214a and 214b located at both ends of the open link structure 200 have, at their respective ends, electrode pads 201a and 201b used for electric connection or signal extraction, the electrode pads 201a and 201b each corresponding to the conductive layer of the FCB 201 exposed to the outside.

A space between the link 211 and the link 212, a space between the link 212 and the link 213, a space between the link 213 and the link 214a, and a space between the link 214b and the link 211 constitute hinge parts 221, 222, 223, and 224 connected by the FCB 201. As described above, since the FCB 201 is a flexible electric circuit board having low rigidity and flexibility, each of the hinge parts 221, 222, 223, and 224 can function as a “joint” that provides a degree of freedom of rotation between links adjacent to each other.

Then, in each of the hinge parts 221, 222, 223, and 224, the conductive layer in the FCB 201 passes through the joint (or a rotation axis), so that it can be said that a wiring structure passing through a hinge is realized. Even when a rotation motion is made between the links, stress such as tension or compression affecting conductivity is kept low, so that an adverse effect on control performance or a risk of cutting wiring is extremely low.

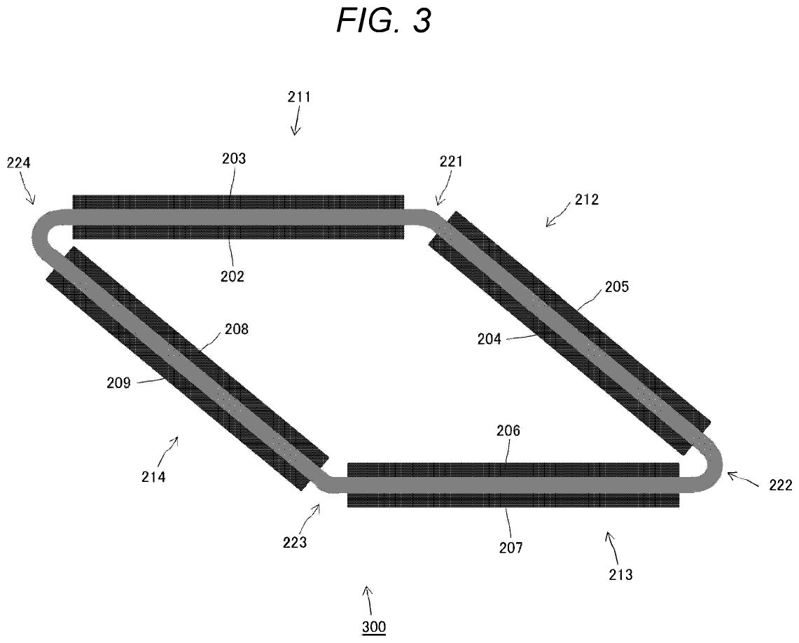

FIG. 3 depicts an example of a closed link structure 300 formed using the FCB 201. The illustrated closed link structure 300 is formed by bending the FCB 201 including the insulating layer, the conductive layer, and the adhesive layer stacked on top of each other of the open link structure 200 depicted in FIG. 2. Then, the respective electrode pads 201a and 201b of the links 214a and 214b located at both ends are bonded together to form a closed link structure including the four links 211 to 214. Here, the links 214a and 214b bonded together are newly defined as a link 214. The link 211 and the link 212 are connected by the hinge 221, the link 212 and the link 213 are connected by the hinge 222, the link 213 and the link 214 are connected by the hinge 223, and the link 214 and the link 211 are connected by the hinge 224. Then, the link 211 and the link 213 facing each other, and the link 212 and the link 214 facing each other are equal in length to each other, so that a parallel link mechanism (or a four-bar link mechanism) can be formed. In this case, when a driving link moves, a driven link moves in the same manner, and angles of the links facing each other are kept identical to each other.

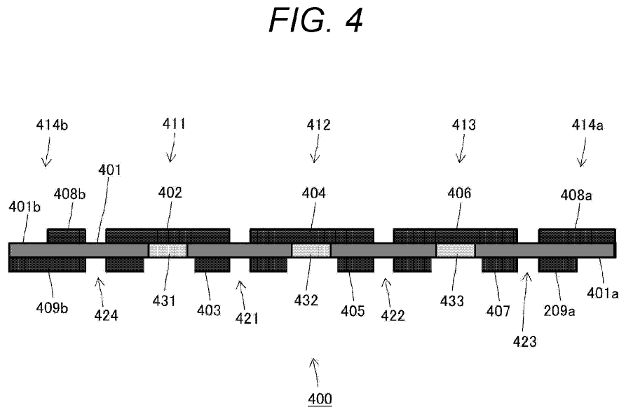

FIG. 4 depicts another example of an open link structure 400 formed using the FCB. In the open link structure 400 in a manner similar to the open link structure 200 depicted in FIG. 2, with a low-rigidity FCB 401 located at a center of the open link structure 400, a link 412 with high rigidity is formed by bonding a pair of high-rigidity parts 402 and 403 to front and back surfaces of the FCB 401, a link 413 with high rigidity is formed by bonding a pair of high-rigidity parts 406 and 407 to the front and back surfaces of the FCB 401, a link 414a with high rigidity is formed by bonding a pair of high-rigidity parts 408a and 409a to the front and back surfaces of the FCB 401, and a link 414b with high rigidity is formed by bonding a pair of high-rigidity parts 408b and 409b to the front and back surfaces of the FCB 401. In FIG. 4, the FCB 401 is depicted in a simplified manner, but actually has a cross-sectional structure as depicted in FIG. 1. Furthermore, the open link structure 400 is further similar to the open link structure 200 in that the open link structure 400 has electrode pads 401a and 401b provided at respective ends of the links 414a and 414b located at both ends of the open link structure 400. Then, a space between the link 411 and the link 412, a space between the link 412 and the link 413, a space between the link 413 and the link 414a, and a space between the link 414b and the link 411 constitute hinge parts 421, 422, 423, and 424 connected by the FCB 401.

Note that the open link structure 400 is different from the open link structure 200 in that the high-rigidity part 403 has an opening in its center to expose the conductive layer of the FCB 401 to the outside through the opening so that the link 411 has an electrode pad 431 used for electric connection or signal extraction, the high-rigidity part 405 has an opening in its center to expose the conductive layer of the FCB 401 to the outside through the opening so that the link 412 has an electrode pad 432 used for electric connection or signal extraction, and the high-rigidity part 407 has an opening in its center to expose the conductive layer of the FCB 401 to the outside through the opening so that the link 413 has an electrode pad 433 used for electric connection or signal extraction.

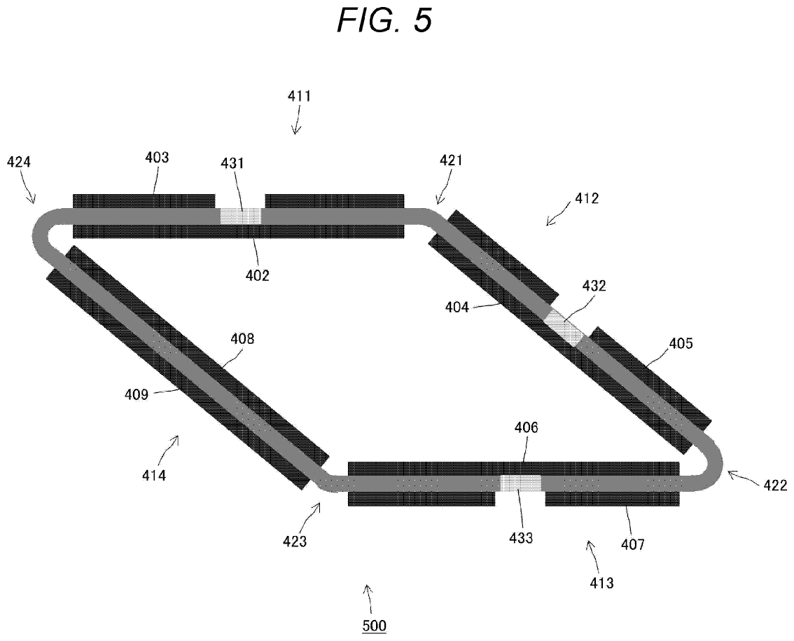

Furthermore, FIG. 5 depicts an example of a closed link structure 500 formed using the FCB 401. The illustrated closed link structure 500 corresponds to a closed link structure formed by bending the FCB 401 constituting the open link structure 400 depicted in FIG. 4 and bonding the respective electrode pads 401a and 401b of the links 414a and 244b located at both ends of the open link structure 400 together. Then, the links 414a and 414b bonded together are newly defined as a link 414.

In the closed link structure 500 in a manner similar to the closed link structure 300 depicted in FIG. 3, the link 411 and the link 413 facing each other, and the link 412 and the link 414 facing each other are equal in length to each other, so that a parallel link mechanism (or a four-bar link mechanism) can be formed. In this case, when a driving link moves, a driven link moves in the same manner, and angles of the links facing each other are kept identical to each other. Note that the open link structure 500 is different from the open link structure 300 in that the links 411, 412, and 413 have the electrode pads 431, 422, and 433 used for electric connection or signal extraction, respectively.

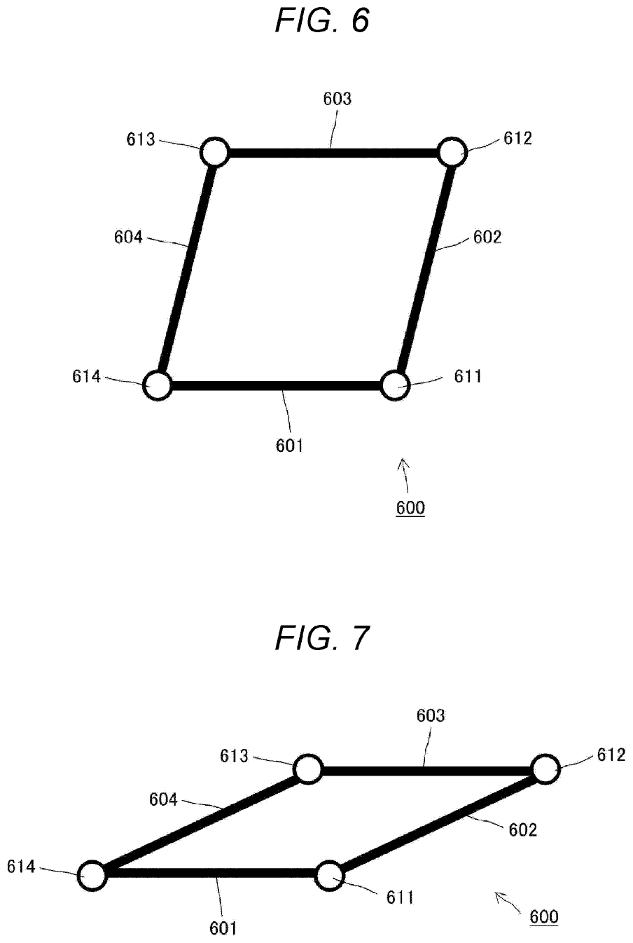

FIG. 6 schematically depicts a degree-of-freedom configuration of the closed link structures depicted in FIGS. 3 and 5. A degree-of-freedom configuration model 600 depicted in FIG. 6 includes four links 601 to 604 and four joints 611 to 614 each connecting links adjacent to each other. The links 601 to 604 and the joints 611 to 614 are arranged with a low-rigidity FCB located at the center, and the links 601 to 604 each include high-rigidity parts including a high-rigidity material bonded to both the front and back sides of the FCB.

An angle between links adjacent to each other changes when a portion including only the FCB between the links is bent. Each of the joints 611 to 614 includes only the FCB, in other words, the conductive layer in the FCB passes through the rotation axis, so that a wiring structure passing through a hinge is realized. Each of the joints 611 to 614 can be regarded as a driven joint having a degree of freedom of rotation about an axis orthogonal to the page.

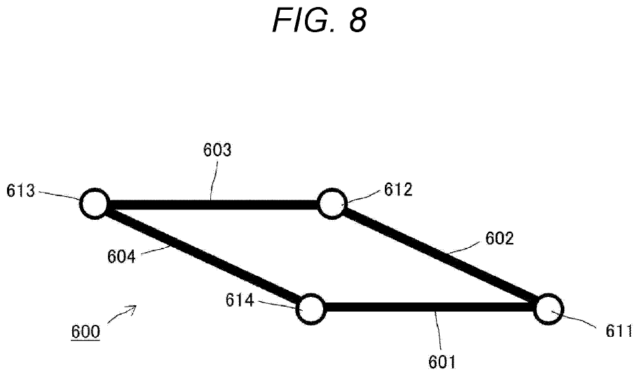

Then, the link 601 and the link 603 facing each other, and the link 602 and the link 604 facing each other are equal in length to each other, so that the degree-of-freedom configuration model 600 constitutes a parallel link mechanism (or a four-bar link mechanism). In this case, when a driving link moves, a driven link moves in the same manner, and angles of the links facing each other are kept identical to each other. FIGS. 7 and 8 depicts states where the link 601 is used as a fixed link, and the link 602 as a driving link and the link 604 as a driven link rotate clockwise and counterclockwise.

### C. Configuration of Manipulator

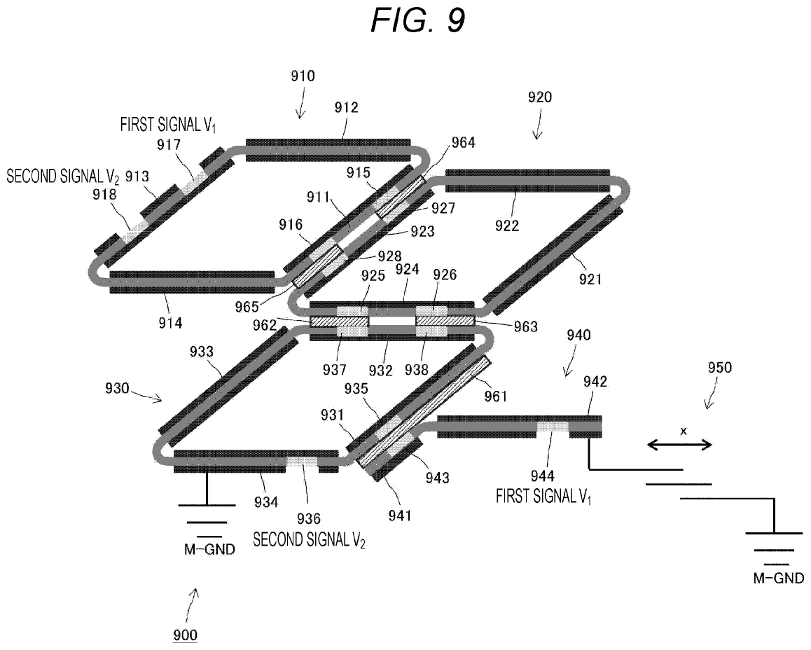

FIG. 9 depicts a configuration example of a manipulator 900 having a parallel link structure including a plurality of closed link structures coupled to each other, the plurality of closed link structures having at least some links provided with an electrode pad as depicted in FIG. 5.

A closed link structure 910, a closed link structure 920, and a closed link structure 930 are coupled in this order from a distal end of the manipulator 900. One link 934 of the closed link structure 930 located at a proximal end side serves as a mechanical ground (or a fixed link).

A link 941 of an open link structure 940 is coupled to a link 931 hinge-coupled to one end of the link 934. Furthermore, a link 942 of the open link structure 940 can be moved in a horizontal direction of the page (or x direction) by a linear motion actuator 950 having one end serving as the mechanical ground. Therefore, the link 931 serves as a driving link. Furthermore, a link 933 facing the link 931 serves as a driven link, and the other link 932 serves as an intermediate link.

Note that specific configurations of each of the closed link structures 910 to 930 and the open link structure 940 are similar to the configurations depicted in FIGS. 1 to 6, so that no detailed description will be given here.

The open link structure 940 has one electrode pad 943 in the link 942 and one electrode pad 944 in the link 941. The electrode pad 943 is used to input and output a first signal V₁, and the electrode pad 944 is used to transmit the first signal V₁ to and from the closed link structure 930.

The link 931 of the closed link structure 930 has one electrode pad 935 at a position facing the electrode pad 944. Then, the link 941 of the open link structure 940 is fixed to the link 931 of the closed link structure 930 with conduction between the electrode pad 944 and the electrode pad 935 established by a joining part 961 having conductivity. Therefore, the first signal V₁ can be transmitted between the closed link structure 930 and the open link structure 940. Furthermore, the closed link structure 930 has one electrode pad 936 in the link 934. The electrode pad 936 is used to input and output a second signal V₂.

The closed link structure 930 has two electrode pads 937 and 938 in the link 932, the electrode pads 937 and 938 being used for the first signal and the second signal, respectively. Furthermore, a link 924 of the closed link structure 920 coupled to the link 932 has two electrode pads 925 and 926 at positions facing the electrode pads 937 and 938, respectively. Then, the link 924 is fixed to the link 932 with conduction between the electrode pad 925 and the electrode pad 937 and conduction between the electrode pad 926 and the electrode pad 938 established, respectively, by joining parts 962 and 963 having conductivity. Therefore, the first signal V₁ and the second signal V₂ can be transmitted between the closed link structure 930 and the closed link structure 920.

The closed link structure 920 has two electrode pads 927 and 928 in a link 923, the electrode pads 927 and 928 being used for the first signal V₁ and the second signal V₂, respectively. Furthermore, a link 911 of the closed link structure 910 coupled to the link 923 has two electrode pads 915 and 916 at positions facing the electrode pads 927 and 928, respectively. Then, the link 911 is fixed to the link 922 with conduction between the electrode pad 915 and the electrode pad 927 and conduction between the electrode pad 916 and the electrode pad 928 established, respectively, by joining parts 964 and 965 having conductivity. Therefore, the first signal V₁ and the second signal V₂ can be transmitted between the closed link structure 920 and the closed link structure 910.

A link 913 of the closed link structure 911 corresponds to a link located at the distal end of the manipulator 900, and constitutes a portion to which an end effector including a surgical instrument such as forceps (not depicted in FIG. 9) is attached. Then, the link 913 has two electrode pads 917 and 918 used for the first signal V₁ and the second signal V₂, respectively. Therefore, the first signal V₁ and the second signal V₂ can be transmitted between the manipulator 900 and the end effector attached to the distal end of the manipulator 900.

The surgical instrument that is used with the surgical instrument attached to the manipulator 900 includes a memory that stores, for example, a surgical instrument identification ID for identifying the type, specification, capabilities, or individual information of the surgical instrument, authentication information used for determining whether or not the surgical instrument is usable on the manipulator 900, calibration data for operation of the surgical instrument, and the like. Then, the manipulator 900 can access the surgical instrument through an electric interface including the electrode pads 917 and 918 located at the distal end of the manipulator 900, read the surgical instrument identification ID from the memory, and transmit corresponding authentication information, calibration data, and the like to the memory in the surgical instrument.

The manipulator 900 according to the present embodiment has a wiring structure in which a signal line used for transmission of the first signal V₁ and the second signal V₂ passes through a hinge. Therefore, even when the manipulator 900 is operated to make a rotation motion between the links, stress such as tension or compression affecting conductivity is kept low, so that an adverse effect on control performance or a risk of cutting wiring is extremely low.

On the signal transmission line, a control signal and power to the surgical instrument that is the end effector, a signal of information read from the memory in the surgical instrument, and the like are transmitted.

Note that FIG. 9 depicts an example where the manipulator 900 has a 2-bit wide signal transmission line for the first signal V₁ and the second signal V₂, but the bit width of the signal transmission line can be easily increased to 3 bits or more.

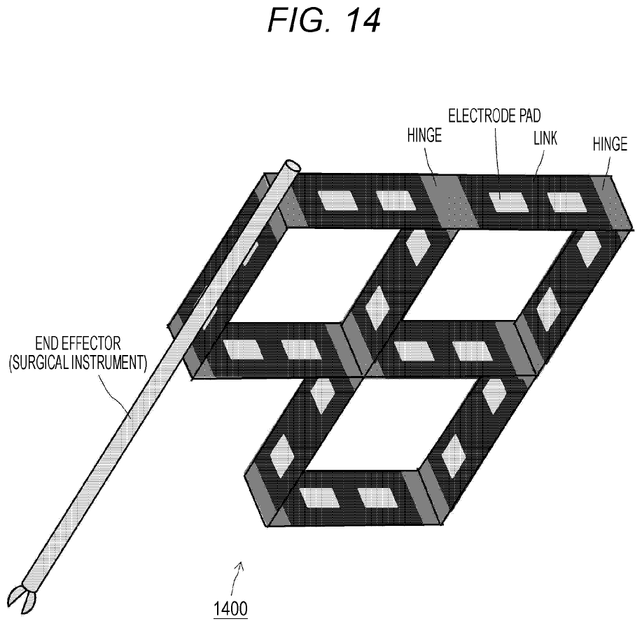

FIG. 9 depicts, for convenience of description, a plan view of the manipulator 900 as viewed from right beside, and each link is depicted like a wire. In practice, since each link includes the FCB as a base member, the link is a rigid body having a uniform width. FIG. 14 depicts a three-dimensional image example of a manipulator 1400 that is identical in degree of freedom to the manipulator 900 depicted in FIG. 9.

FIG. 14 depicts a state where an end effector including a surgical instrument such as forceps is attached to a link located at the distal end of the manipulator 1400. Wiring can be easily routed from the portion to which the end effector is attached to the mechanical ground. In particular, providing no aerial wiring around the end effector makes the separation of the clean region and the non-clean region and the cleaning and sterilization work easier. Furthermore, providing the opening in the high-rigidity part attached to the link portion allows the electrode pad for inputting and outputting the electric signal to be provided at any position in the manipulator, so that the degree of freedom of mechanical design is improved.

### D. Kinematics of Manipulator

In this section D, kinematics of the manipulator 900 described in the above-described section C with reference to FIG. 9 will be described.

In a case where the surgical instrument attached to the distal end of the manipulator 900 is operated to perform a surgical operation, it is necessary to perform, for minimum invasiveness, the operation with a load as small as possible on the vicinity of a trocar into which the surgical instrument is inserted, so that it is ideal to cause the surgical instrument to pivot using the trocar insertion point as a fulcrum (or with the trocar insertion point fixed) to make an impulse generated at the trocar insertion point equal to zero.

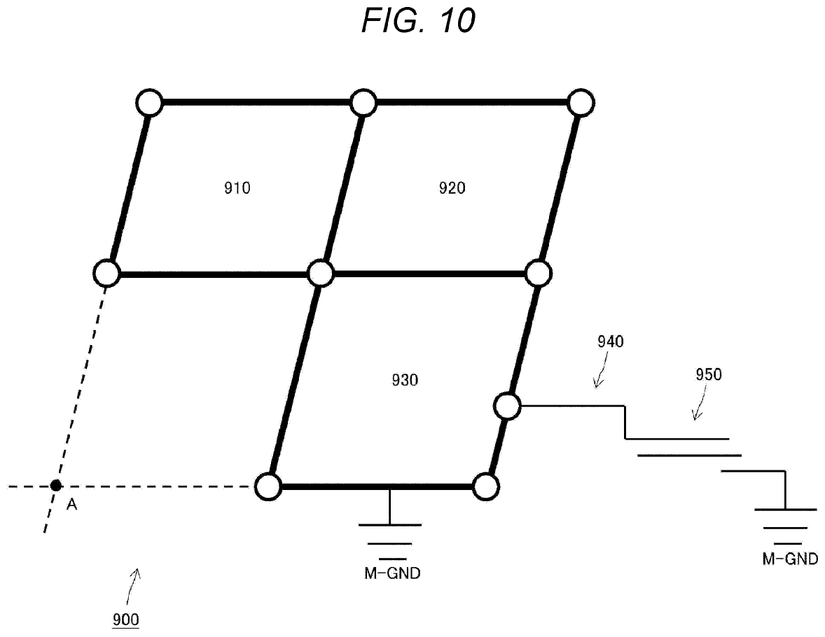

FIG. 10 depicts a degree-of-freedom configuration model of the manipulator 900 depicted in FIG. 9. Note that, in FIG. 10, each high-rigidity link is drawn by a thick line, and a hinge portion connecting the links is indicated by a circle coaxial with the rotation axis. Furthermore, a link serving as a joining portion between closed link structures adjacent to each other is also drawn by one thick line for the sake of simplification.

In FIG. 10, the axis of the link (fixed link) 934 of the closed link structure 930 and the axis of the link 913, to which the surgical instrument is attached, of the closed link structure 910 located at the distal end intersect at a point A.

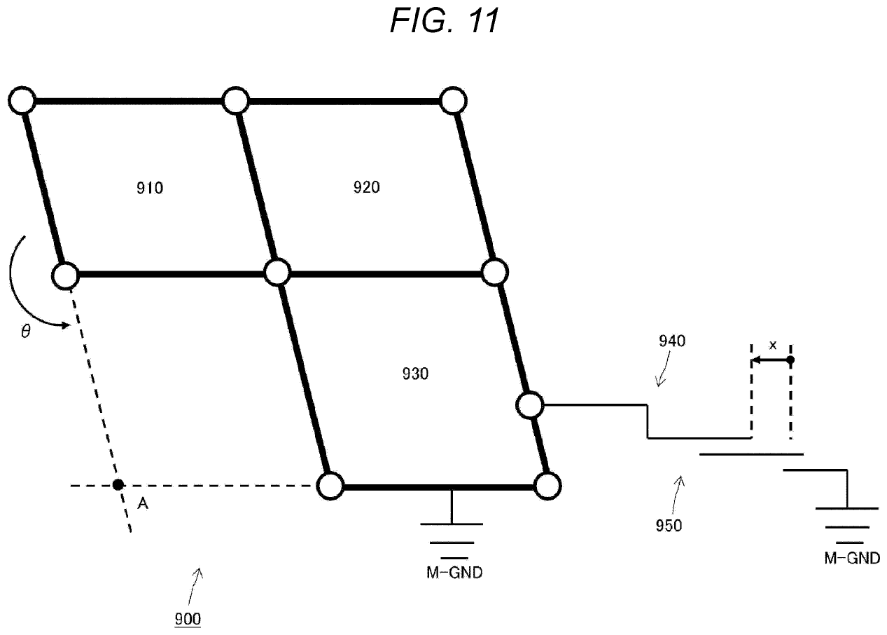

FIG. 11 depicts a state where moving the linear motion actuator 950 in the x direction rotates the link 931 serving as a driving link of the closed link structure 930 in a counterclockwise direction of the page via the open link structure 940. Assuming that each link of the other closed link structure 920 and the closed link structure 910 is kept in parallel with a corresponding link of the closed link structure 930, the axis of the link (fixed link) 934 of the closed link structure 930 and the axis of the link 913, to which the surgical instrument is attached, of the closed link structure 910 located at the distal end also intersect at the point A. That is, the intersection point A is a fixed point.

It is therefore possible to achieve, by setting the trocar insertion point at the intersection point A, minimally invasive surgery using the surgical instrument attached to the link 913. E. Usage Example of Surgical Instrument

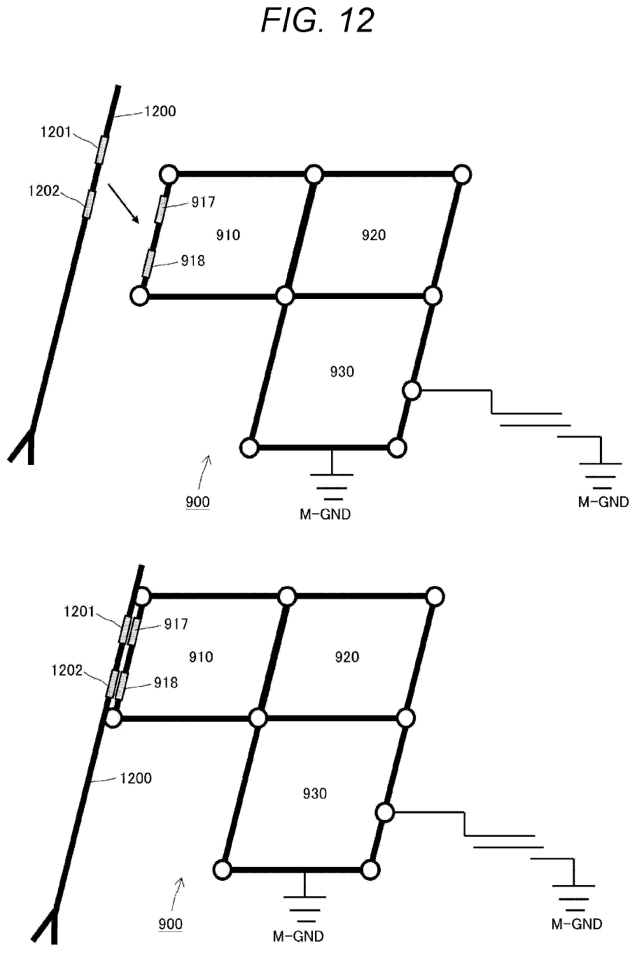

FIG. 12 depicts an example where the surgical instrument is used with the surgical instrument attached to the manipulator 900. The illustrated surgical instrument 1200 includes two electrode pads 1201 and 1202 each electrically connected to a corresponding one of the electrode pads 917 and 918 of the link 913 located at the distal end of the manipulator 900. Therefore, the first signal V₁ and the second signal V₂ can be transmitted between the surgical instrument 1200 and the manipulator 900.

The surgical instrument includes a memory that stores, for example, a surgical instrument identification ID for identifying the type, specification, capabilities, or individual information of the surgical instrument, authentication information used for determining whether or not the surgical instrument is usable on the manipulator 900, calibration data for operation of the surgical instrument, the date of manufacture, and the like. Then, the manipulator 900 can access the surgical instrument through the electric interface including the electrode pads 917 and 918 located at the distal end of the manipulator 900, read the surgical instrument identification ID from the memory, and transmit corresponding authentication information, calibration data, and the like to the memory in the surgical instrument.

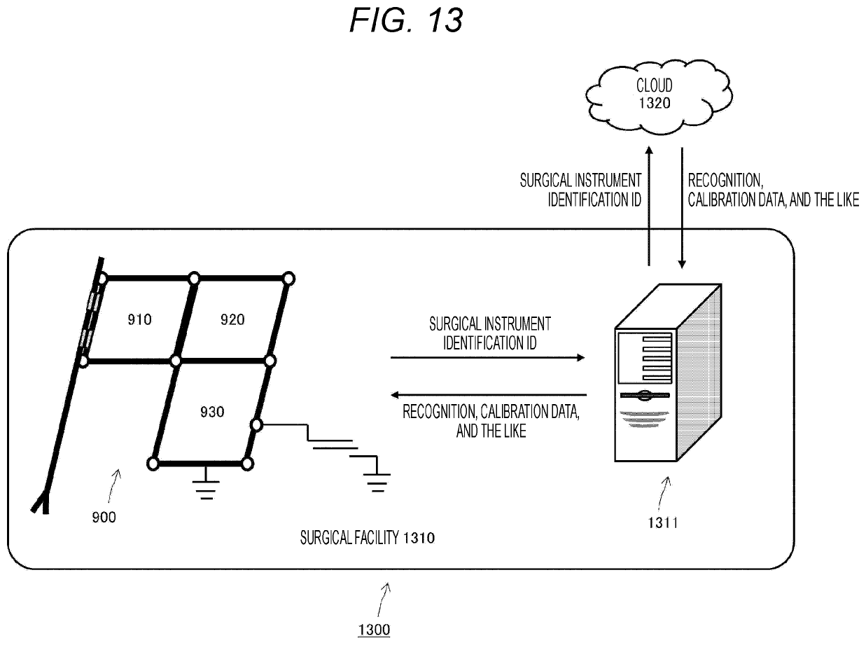

FIG. 13 depicts a configuration example of a system 1300 in which the manipulator 900 performs authentication of the surgical instrument 1200 attached to the distal end of the manipulator 900 and acquires the surgical instrument information such as the calibration data.

In a surgical facility 1310 such as a hospital, the manipulator 900 to which the surgical instrument is attached and an authentication server 1311 that performs authentication processing on the surgical instrument attached to the manipulator 900 are arranged.

The manipulator 900 transfers, to the authentication server 1311, the surgical instrument identification ID read from the surgical instrument attached to the manipulator 900.

The authentication server 1311 uploads the surgical instrument identification ID acquired from the manipulator 900 to a cloud 1320 and intervenes between the cloud 1320 and the manipulator 900 to perform the authentication processing on the surgical instrument.

Then, when the authentication processing results in a success, the authentication server 1311 downloads the calibration data of the surgical instrument from the cloud 1320 and transfers the data to the manipulator 900. The manipulator 900 transmits the calibration data received from the authentication server 1311 to the surgical instrument located at the distal end through the transmission line of the first signal and the second signal to write the calibration data to the memory in the surgical instrument. As a result, the manipulator 900 is brought into a state where the manipulator 900 can perform a surgical operation using the surgical instrument.

### F. Effects

In this section F, effects brought about by the manipulator to which the present disclosure is applied will be described.
(1) According to the present disclosure, it is possible to make routing of wiring from the portion, to which the end effector is attached, at the distal end of the manipulator to the mechanical ground easier. In particular, in a case where the manipulator is applied to a surgical robot, providing no aerial wiring around the end effector makes the separation of the clean region and the non-clean region and the cleaning and sterilization work easier. Furthermore, providing the opening in the high-rigidity part attached to the link portion allows the electrode pad for inputting and outputting the electric signal to be provided at any position in a robot arm, so that the degree of freedom of mechanical design is improved. The electric signal is input and output at any position in the robot arm
(2) In general, it is necessary for a surgical robot to change a plurality of types of surgical instruments during one surgery. According to the present disclosure, it is possible to read information such as the surgical instrument identification ID retained in the memory in the surgical instrument to perform the authentication processing, or acquire information such as corresponding configuration data on the basis of the surgical instrument identification ID to write the information to the memory of the surgical instrument or transmit the information to a control computer of the surgical robot.

### G. Modification Example

In this section G, the above-described embodiment will be described.
(1) In the above-described embodiment, the wiring laid in the FCB serving as the base member of the manipulator is electric wiring, but the signal transmission medium is not limited to any specific medium. For example, the manipulator may include, as the base member, an FCB in which an optical fiber is laid. For example, it is possible to measure, by measuring a strain on the link with a fiber optic strain sensor such as a fiber bragg grating (FBG) provided in the hinge portion, a strain generated on the robot arm when an external force is applied to the distal end of the end effector and estimate the external force on the basis of the measurement result.
(2) It is possible to form an electric circuit for control or other applications directly on the robot arm by mounting electric components such as a chip resistor and an integrated circuit (IC) on the FCB serving as the base member.
(3) For signal input/output of the FCB, a non-contact antenna or the like may be used instead of an electric pad.
(4) A cloud (or a data server installed outside the surgical facility) may transmit authentication data or configuration data of the surgical instrument attached to the distal end of the robot arm.

### INDUSTRIAL APPLICABILITY

The present disclosure has been described in detail with reference to the specific embodiment. It is, however, obvious that those skilled in the art can make modifications and substitutions of the embodiment without departing from the gist of the present disclosure.

The present disclosure is applicable mainly to ocular surgery such as retinal surgery, and is further applicable to various types of surgery performed with a surgical instrument inserted into a body through a trocar. Furthermore, the present disclosure is also applicable to, for example, remote control or operation support using a master-slave robot, or autonomous control of a surgical robot.

Furthermore, examples of the surgical instrument attached to the manipulator according to the present disclosure may include, other than the forceps, a tweezer, an insufflation tube, an energy treatment tool, and a medical observation device such as a microscope and an endoscope (a rigid endoscope such as a laparoscope and an arthroscope, and a flexible endoscope such as a gastrointestinal endoscope and a bronchoscope).

In short, the present disclosure has been described in an illustrative manner, and the contents described herein should not be interpreted in a limited manner. In order to determine the gist of the present disclosure, the claims should be taken into consideration.

Note that, the present disclosure may also have the following configurations.
(1) A robot device including:
   a plurality of links; and
   a hinge part including a deformable signal transmission part and connecting the links adjacent to each other.
(2) The robot device described in (1), further including a flexible circuit board including a signal transmission line layer and a low-rigidity insulating layer stacked on top of each other, the signal transmission line layer transmitting a signal, the low-rigidity insulating layer insulating the signal transmission line layer,
   in which
   each of the plurality of links is formed by the flexible circuit board having a high-rigidity material bonded to both sides or at least one side thereof, and
   the hinge part is formed by the flexible board having no high-rigidity material bonded to either of the sides thereof.
(3) The robot device described in (2), in which the signal transmission line layer includes a conductive layer that transmits an electric signal.
(4) The robot device described in (3), further including an open link structure,
   in which the open link structure includes an electrode pad used for transmission and reception of the electric signal provided at both ends of the flexible board, the electrode pad being formed by an exposed portion of the signal transmission line layer.
(5) The robot device described in (3), further including a closed link structure,
   in which at least some of the high-rigidity materials bonded to the links have an opening, and the closed link structure includes an electrode pad used for transmission and reception of the electric signal, the electrode pad being formed by a portion of the signal transmission line layer exposed through the opening.
(6) The robot device described in (5), further including a plurality of the closed link structures coupled to each other.
(7) The robot device described in (6), in which a link located at a distal end includes an electrode pad used for transmission and reception of the electric signal to and from an end effector attached to the distal end, the electrode pad being formed by a portion of the signal transmission line layer exposed through the opening provided through the bonded high-rigidity material.
(8) The robot device described in (6) or (7), in which power is transmitted to at least some of the links.
(9) The robot device described in (8), in which
   some of the links of the closed link structure located away from a distal end serve as a mechanical ground, and
   the power is transmitted to a link adjacent to the mechanical ground.
(10) The robot device described in (2), in which the signal transmission line layer includes a layer including an optical fiber that transmits an optical signal.
(11) The robot device described in (10), in which at least some of the hinge parts include a fiber optic strain sensor.
(12) A surgical manipulator including:
   a surgical instrument; and
   a link structure including a plurality of links and a hinge part including a deformable signal transmission part and connecting the links adjacent to each other, in which the surgical instrument is attached to a link located at a distal end.
(13) The surgical manipulator described in (12), in which the link structure causes the surgical instrument to pivot with a predetermined trocar insertion point on an axis of the surgical instrument fixed.
(14) A system including:
   a robot device including a plurality of links and a hinge part that includes a deformable signal transmission part and connects the links adjacent to each other, an end effector being attached to a link located at a distal end; and
   an authentication server configured to perform authentication of the end effector,
   in which
   the robot device transmits identification information read from the end effector via the signal transmission part to the authentication server, and
   the authentication server performs authentication processing on the end effector on the basis of the identification information received from the robot device, and acquires configuration data for the end effector.

### REFERENCE SIGNS LIST

100 Electric circuit board (FCB)
200 Open link structure
201 FCB
201a, 201b Electrode pad
202 to 207, 208a, 208b, 209a, 209b High-rigidity part
211 to 214, 214a, 214b Link
221 to 224 Hinge
300 Closed link structure
400 Open link structure
401 FCB
202 to 207, 208a, 208b, 209a, 209b High-rigidity part
411 to 414, 414a, 414b Link
421 to 424 Hinge
431 to 433 Electrode pad
500 Closed link structure
600 Degree-of-freedom configuration model of closed link structure
601 to 604 Link
611 to 614 Joint
900 Manipulator
910 Closed link structure
910 Closed link structure
911 to 914 Link
915 to 918 Electrode pad
920 Closed link structure
921 to 924 Link
925 to 928 Electrode pad
930 Closed link structure
931 to 934 Link
935 to 938 Electrode pad
940 Open link structure
941, 942 Link
943, 944 Electrode pad
950 Linear motion actuator
961 to 965 Joining part
1200 Surgical instrument
1201, 1202 Electrode pad

1. A robot device comprising:
a plurality of links; and
a hinge part including a deformable signal transmission part and connecting the links adjacent to each other.

2. The robot device according to claim 1, further comprising a flexible circuit board including a signal transmission line layer and a low-rigidity insulating layer stacked on top of each other, the signal transmission line layer transmitting a signal, the low-rigidity insulating layer insulating the signal transmission line layer,
wherein
each of the plurality of links is formed by the flexible circuit board having a high-rigidity material bonded to both sides or at least one side thereof, and
the hinge part is formed by the flexible board having no high-rigidity material bonded to either of the sides thereof.

3. The robot device according to claim 2, wherein the signal transmission line layer includes a conductive layer that transmits an electric signal.

4. The robot device according to claim 3, further comprising an open link structure,
wherein the open link structure includes an electrode pad used for transmission and reception of the electric signal provided at both ends of the flexible board, the electrode pad being formed by an exposed portion of the signal transmission line layer.

5. The robot device according to claim 3, further comprising a closed link structure,
wherein at least some of the high-rigidity materials bonded to the links have an opening, and the closed link structure includes an electrode pad used for transmission and reception of the electric signal, the electrode pad being formed by a portion of the signal transmission line layer exposed through the opening.

6. The robot device according to claim 5, further comprising a plurality of the closed link structures coupled to each other.

7. The robot device according to claim 6, wherein a link located at a distal end includes an electrode pad used for transmission and reception of the electric signal to and from an end effector attached to the distal end, the electrode pad being formed by a portion of the signal transmission line layer exposed through the opening provided through the bonded high-rigidity material.

8. The robot device according to claim 6, wherein power is transmitted to at least some of the links.

9. The robot device according to claim 8, wherein
some of the links of the closed link structure located away from a distal end serve as a mechanical ground, and
the power is transmitted to a link adjacent to the mechanical ground.

10. The robot device according to claim 2, wherein the signal transmission line layer includes a layer including an optical fiber that transmits an optical signal.

11. The robot device according to claim 10, wherein at least some of the hinge parts include a fiber optic strain sensor.

12. A surgical manipulator comprising:
a surgical instrument; and
a link structure including a plurality of links and a hinge part including a deformable signal transmission part and connecting the links adjacent to each other, wherein the surgical instrument is attached to a link located at a distal end.

13. The surgical manipulator according to claim 12, wherein the link structure causes the surgical instrument to pivot with a predetermined trocar insertion point on an axis of the surgical instrument fixed.

14. A system comprising:
a robot device including a plurality of links and a hinge part that includes a deformable signal transmission part and connects the links adjacent to each other, an end effector being attached to a link located at a distal end; and
an authentication server configured to perform authentication of the end effector,
wherein
the robot device transmits identification information read from the end effector via the signal transmission part to the authentication server, and
the authentication server performs authentication processing on the end effector on a basis of the identification information received from the robot device, and acquires configuration data for the end effector.
